(19) United States
(12) Patent Application Publication
(10) Pub. No.: US 2024/0180721 A1
(43) Pub. Date: Jun. 6, 2024

(54) FINGER WITH AUTOMATIC MAINTENANCE OF THE GRIPPING POSITION

(71) Applicant: EPIC INVENTING, INC., Los Angeles, CA (US)

(72) Inventor: Mark Hunter, Los Angeles, CA (US)

(21) Appl. No.: 18/284,768

(22) PCT Filed: Apr. 12, 2022
(86) PCT No.: PCT/IB2022/053429

§ 371 Date: Sep. 28, 2023

Related U.S. Application Data
(60) Provisional application No. 63/173,530, filed on Apr. 12, 2021.

(30) Foreign Application Priority Data
Apr. 16, 2021 (GB) 2105448.1

(51) Int. Cl. A61F 2/58; A61F 2/50; A61F 2/70

(52) U.S. Cl. CPC A61F 2/586; A61F 2002/5038; A61F 2002/701; A61F 2002/704

(57) ABSTRACT

A bionic digit (1) that comprises an intermediate portion (15), a tip portion (23) and a hinge (27.29) connecting the tip portion (23) to the intermediate portion (15). The bionic digit (1) further comprises a linear actuator assembly (45) located within the intermediate portion (15) that is connected to the intermediate portion (15) and to the tip portion (23). The linear actuator assembly (45) is provided with a force generator (47), to which is connected a rotary drive shaft (49), and a ball screw (51) connected to the rotary drive shaft (49) for rotation therewith, wherein the ball screw (51) has a helical drive ball raceway (65) extending around its external surface, along at least part of its length, a plurality of drive balls (57), each drive ball (57) located within the helical drive ball raceway (65) and within a drive ball aperture (63) of a ball retention element (55) that is located around the ball screw (51), that is shorter than the ball screw (51) and that is moveable relative to the ball screw (51). Each drive ball (57) is also located within an annular groove (77) of a drive collar (53) that is positioned around the ball retention element (55), the drive collar (53) being rotatable relative to the ball retention element (55) and the ball screw (51) around the longitudinal axis L-L of the ball screw (51). The drive collar (53) has multiple annular grooves (77) that are parallel to each other and perpendicular to the longitudinal axis of the ball screw (51) and is provided with a first engagement element (79). The tip portion (23) is provided with a second engagement element (83) and the first engagement portion (79) and the second engagement portion (83) are engaged with each other.

The present invention relates generally to electromechanical digits and particularly, but not exclusively, to bionic fingers with a linear actuator assembly, as well as to prosthetic hands including the electromechanical or bionic digits.

Prosthetic hands having one or more moveable bionic digits are well known. For example, WO2015138968 discloses a bionic digit comprising a knuckle, a proximal element, a distal element, a force actuator and a rod. The force generator includes an electrical motor that turns a screw. A threaded nut that is coupled to the screw can be forced to move forward or backward along the axis of the screw as the screw is driven to rotate by the motor. These parts are connected to each other by four pivotal connectors. A first pivotal connector connects the proximal element to the knuckle and a second pivotal connector connects a proximal end of the rod to the knuckle, the first and second connectors being spaced apart. The third pivotal connector connects the threaded nut to both the distal and the proximal elements and to the distal end of the rod. The fourth pivotal connector connects the distal element to the proximal element, for allowing the distal element to pivot relative to the proximal element. The third and fourth connectors being spaced apart. As the threaded nut is driven along the screw axis, it acts at the third connector to force the distal element to pivot relative to the proximal element at the fourth connector. The rod ensures that the threaded nut remains at a predetermined distance from the second connector, causing the proximal element to rotate relative to the knuckle as the threaded nut is driven to move along the screw axis.

For prosthetic hands that are provided with electric motors that drive movement of the bionic digits, i.e. extension and retraction of those digits, it is desirable to stop the supply of electrical power to the electric motors when the digits are to be retained in position for an extended period of time, in order to save electrical energy (typically battery power). For example, the electrical supply to the motors will be turned off when the digits on the prosthetic hand are in a retracted position and grasping the handle of a bag. It may be necessary to retain the hand in a grasped position for a long period of time if the bag is being carried over a substantial distance and whilst the bag is being carried its handles will exert a force on the digits. A component of that force will try to move the digits from their retracted position towards their extended position and, if the force is able to move the digits in that direction then the prosthetic hand will open, the digits grasp of the handles will fail and the bag will drop to the floor. Opening of the digits can occur if the force applied to them is sufficient to drive the linear actuator in the opening direction. This is known as back-driving. Therefore, the digits need to be self-locking.

If the linear actuator is a screw drive with a threaded drive nut that is driven in a linear direction by the rotation of a leadscrew, then if back-driving is to occur the force applied to the digit, must be sufficient to overcome the frictional resistance that exists between the drive nut and the leadscrew. In a leadscrew drive, the amount of frictional resistance is determined by the coefficients of friction of the materials from which the drive nut and leadscrew are made and by the geometry of the drive nut and the leadscrew, for example the pitch of the screw thread. There are two means to prevent back-driving from occurring, use of a brake or use of a leadscrew drive with a high frictional resistance. However, neither of these means are desirable. Use of a brake will add complexity to the digit and thus cost and weight. Use of a high frictional resistance leadscrew drive will reduce the efficiency of operation of the linear actuator because when the leadscrew drive is being driven by the motor in either direction, to extend or retract the digit, the frictional force needs to be overcome before the drive nut will move and thus before the digit can be articulated. Consequently, it has been identified that there is a need for an actuator arrangement that can resist back-driving without comprising the operation of the actuator when it is being driven, for example by an electric motor.

Accordingly the present invention provides a bionic digit comprising an intermediate portion, a tip portion and a hinge connecting the tip portion to the intermediate portion, and further comprising a linear actuator assembly located within the intermediate portion that is connected to the intermediate portion and to the tip portion and that is provided with a force generator, to which is connected a rotary drive shaft, and a ball screw connected to the rotary drive shaft for rotation therewith, wherein the ball screw has a helical drive ball raceway extending around its external surface, along at least part of its length, a plurality of drive balls, each drive ball located within the helical drive ball raceway and within a drive ball aperture of a ball retention element that is located around the ball screw and that is moveable relative to the ball screw, wherein each drive ball is also located within an annular groove of a drive collar that is positioned around the ball retention element, the drive collar being rotatable relative to the ball retention element and the ball screw around the longitudinal axis L-L of the ball screw, wherein the drive collar has multiple annular grooves that are parallel to each other and perpendicular to the longitudinal axis of the ball screw, wherein the drive collar is provided with a first engagement element and the tip portion is provided with a second engagement element and the first and second engagement portions are engaged with each other.

Preferably, the ball screw is provided with a proximal end stop and a distal end stop and wherein the ball retention element is provided with a proximal end stop abutment and a distal end stop abutment.

Preferably, the ball screw has a circular cross-sectional profile, the ball retention element is a cylindrical tube with an internal diameter that is larger than the external diameter of the ball screw and the drive collar has a circular cross-section bore with an internal diameter that is larger than the external diameter of the ball retention element.

Preferably, the helical drive ball raceway has a hemi-spherical cross-sectional profile, wherein when a drive ball is placed within the drive ball raceway there is a clearance between at least some portion of the drive ball and the drive ball raceway. Alternatively, the drive ball raceway can have a V shaped profile.

Preferably, the multiple annular grooves of the drive collar have a V shaped cross-sectional profile and wherein the angle of the V is such that when a drive ball is placed in an annular groove at least a portion of the drive ball extends past the open end of the annular groove.

Preferably, the bionic digit further comprises a base portion attached to the proximal end of the intermediate portion by a base hinge.

Preferably, the pitch of the ball screw is between 0.25 mm and 4 mm.

Preferably, the force generator of the linear actuator assembly is an electric motor and gearbox with a rotor that rotates around an axis coaxial with the longitudinal axis L-L, wherein the drive shaft is attached to the rotor and rotates with the rotor and extends outwardly from the electric motor along longitudinal axis L-L, the driveshaft is located within a recess in the ball screw so that the ball screw can move axially relative to the driveshaft along axis L-L but cannot rotate relative to the driveshaft, the ball screw has a circular cross-section, the retention element is a straight-sided cylindrical tube with a plurality of circular drive ball apertures that pass through the wall of the tube and that are equally angularly spaced apart around the circumference of the tube, wherein the drive collar has a circular internal bore provided with a plurality of annular grooves that are located perpendicularly to longitudinal axis L-L, wherein the first engagement element of the drive collar is a peg that extends perpendicularly from the external surface of the drive collar and perpendicularly to a central plane CP through which longitudinal axis L-L passes, the first engagement element being located within the second engagement element which takes the form of a slot in the proximal end of the tip portion.

According to another aspect of the present invention there is provided a prosthetic hand comprising a plurality of bionic digits according to any one of the preceding claims wherein, the prosthetic hand is provided with a palm, the bionic digits are each provided with a base portion and the base portion of each bionic digit is attached to the palm.

The present invention will be described below, with reference to the accompanying figures:

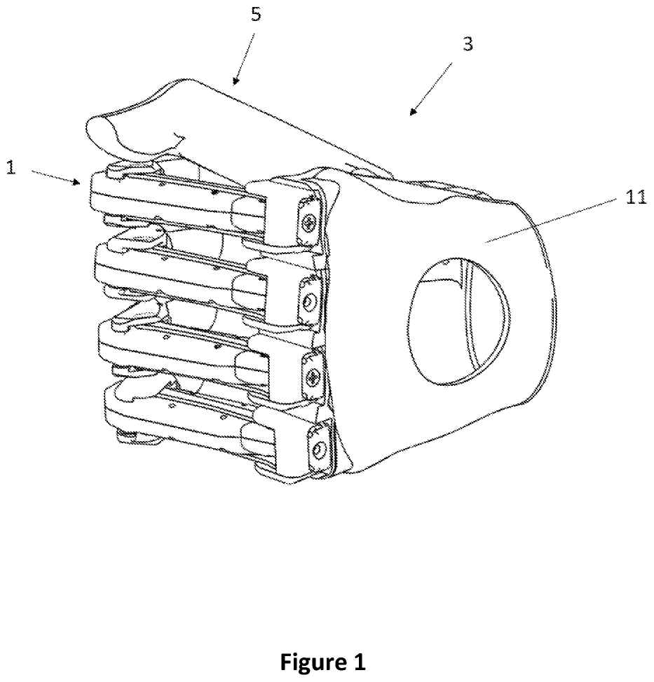

FIG. 1 is a top perspective view of a partially closed prosthetic hand;

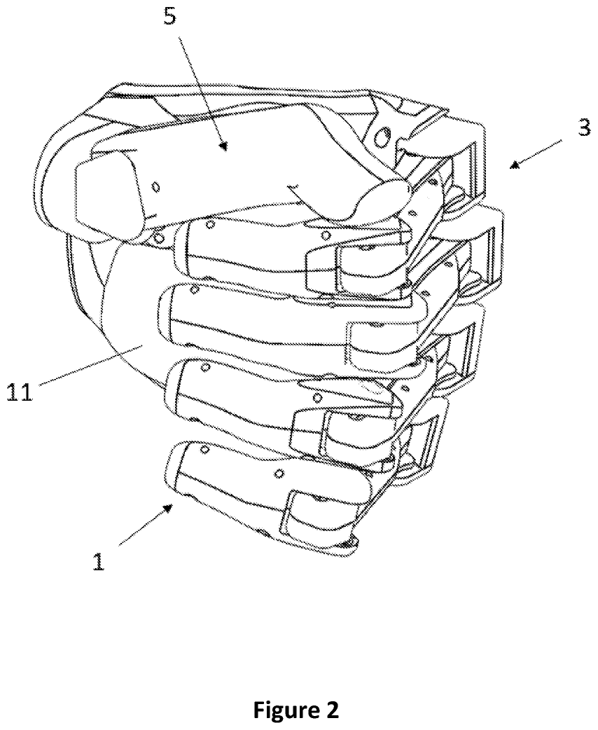

FIG. 2 is perspective view of the underneath of the partially closed prosthetic hand of FIG. 1;

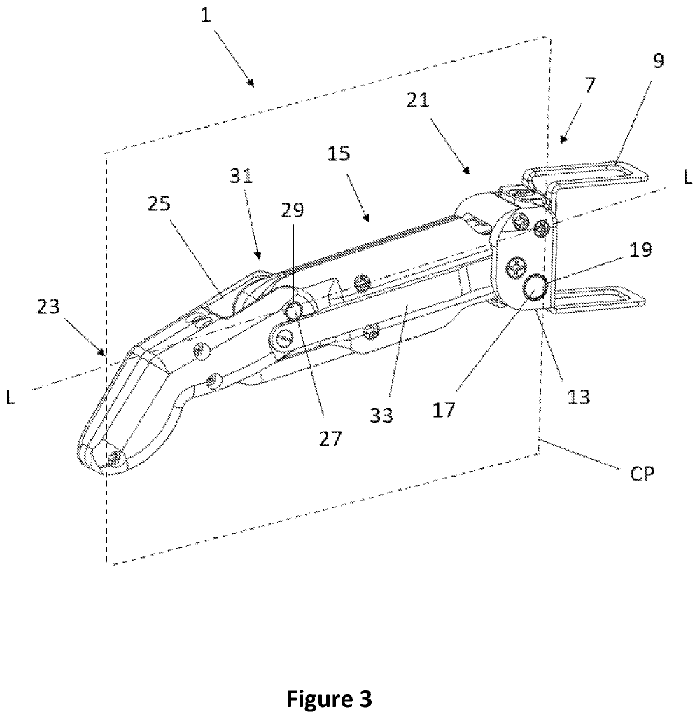

FIG. 3 is a perspective view of an embodiment of a electromechanical, or bionic, finger, such as would be used in the prosthetic hand of FIGS. 1 and 2, in a fully extended, or fully open, position;

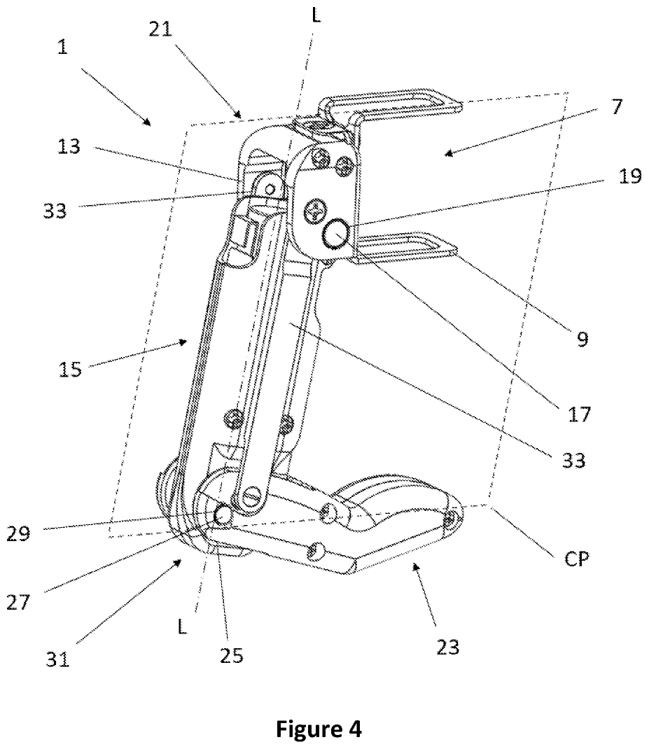

FIG. 4 is a perspective view of the bionic finger of FIG. 3, in a fully retracted, or fully closed, position;

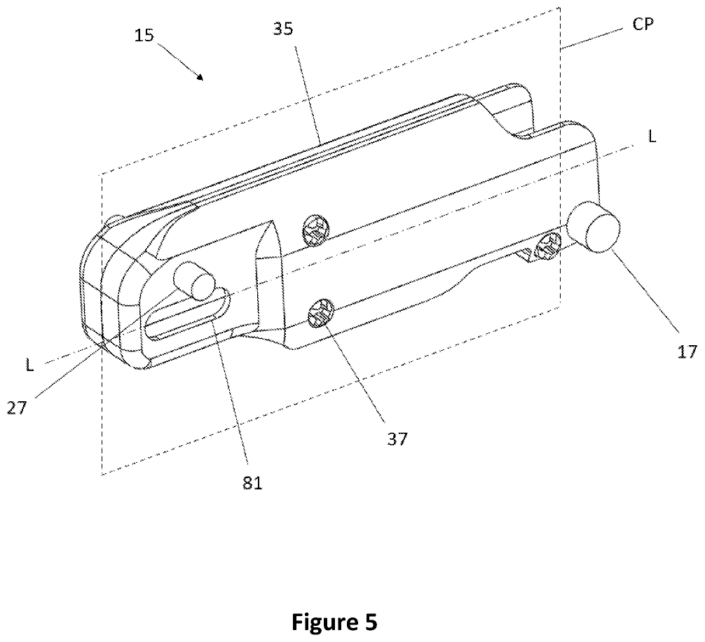

FIG. 5 is a perspective view of the intermediate portion of the bionic finger of FIG. 3;

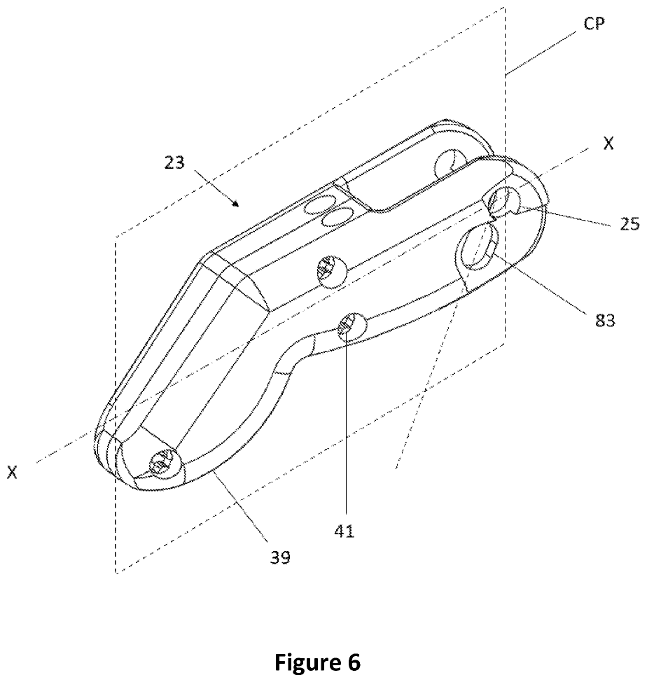

FIG. 6 is a perspective view of the end portion of the bionic finger of FIG. 3;

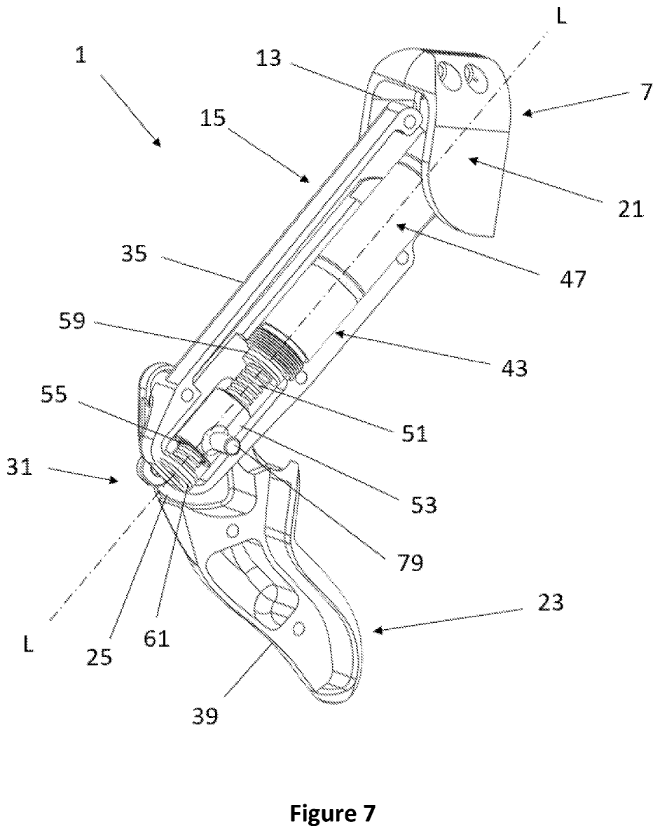

FIG. 7 is perspective cutaway view of the bionic finger, in a partially retracted position, showing the linear actuator assembly;

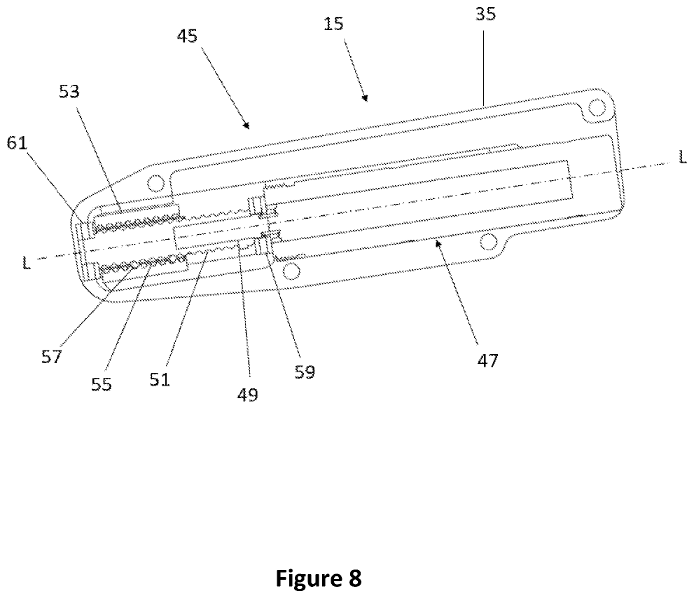

FIG. 8 is a side cross-sectional view of the intermediate portion of FIG. 5 showing the linear actuator assembly;

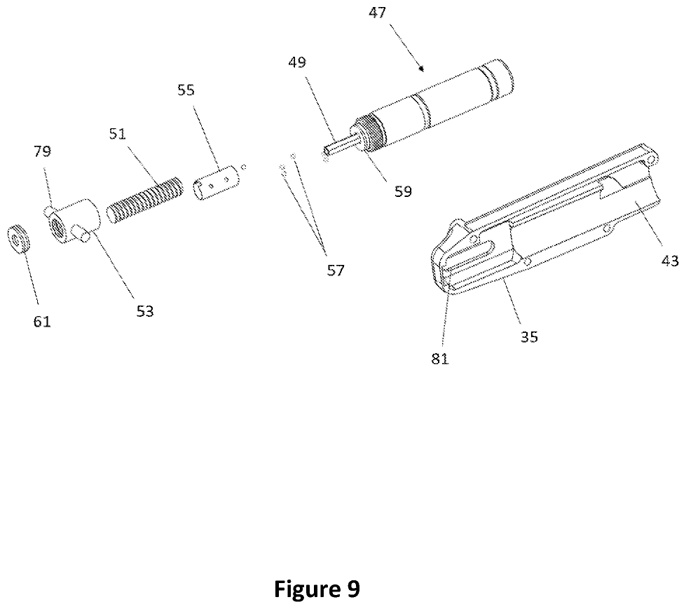

FIG. 9 is a partially exploded perspective view of the intermediate portion of FIG. 5 showing the linear actuator assembly;

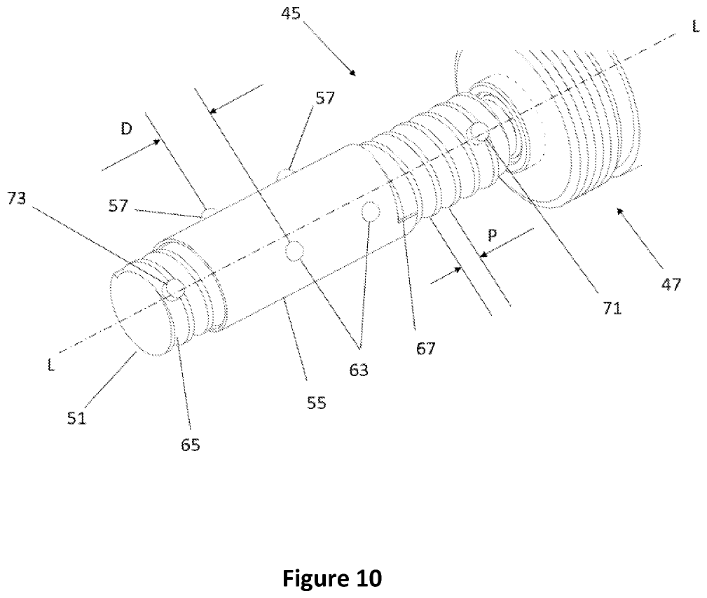

FIG. 10 is a perspective view of the ball cage located around the ball screw;

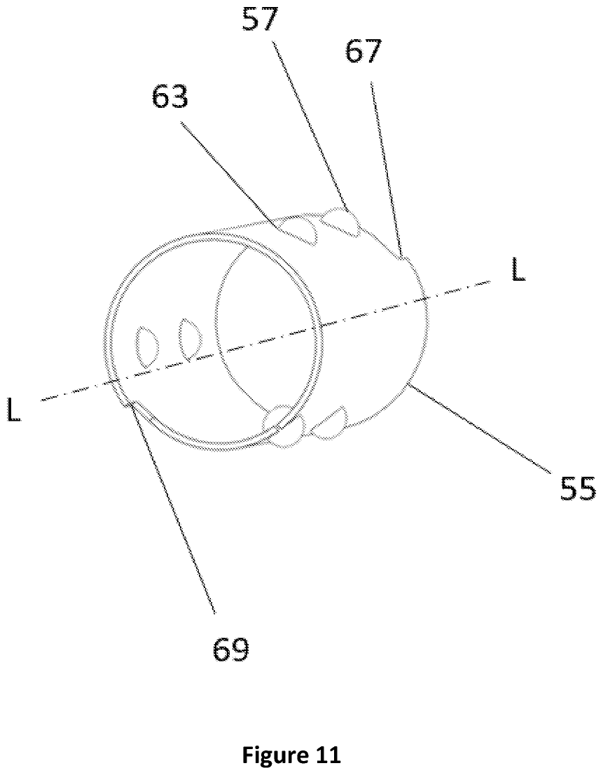

FIG. 11 is an end perspective view of the ball cage;

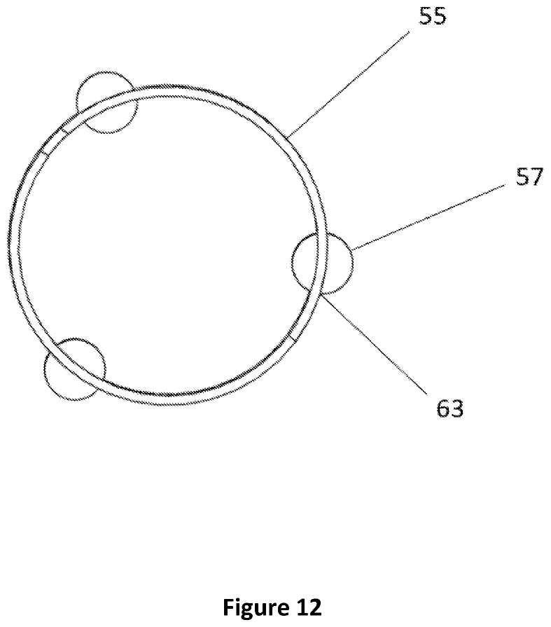

FIG. 12 is an end view of the ball cage;

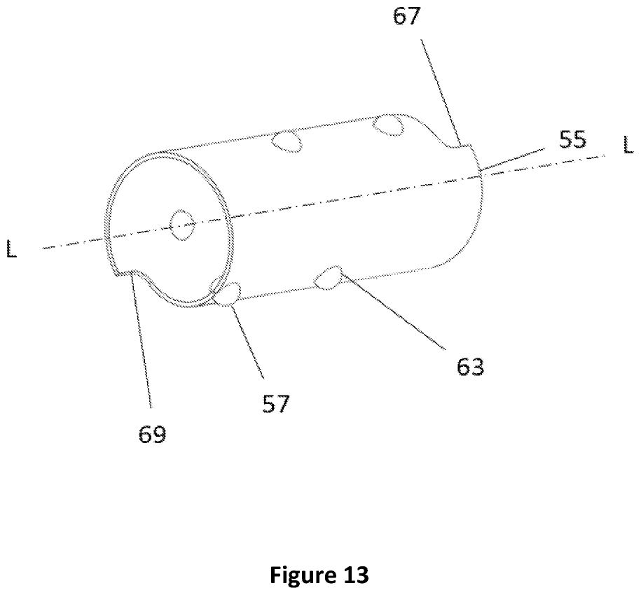

FIG. 13 is a side perspective view of the ball cage;

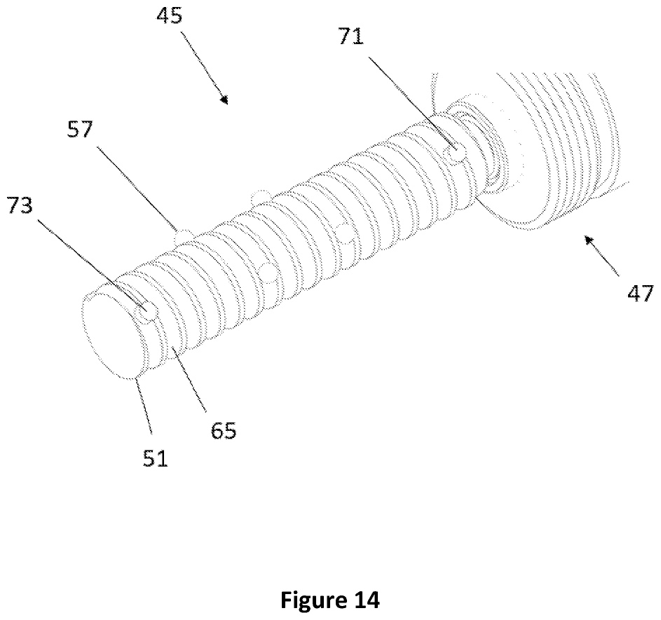

FIG. 14 is a perspective view of the ball screw with end stops and with the drive balls in position;

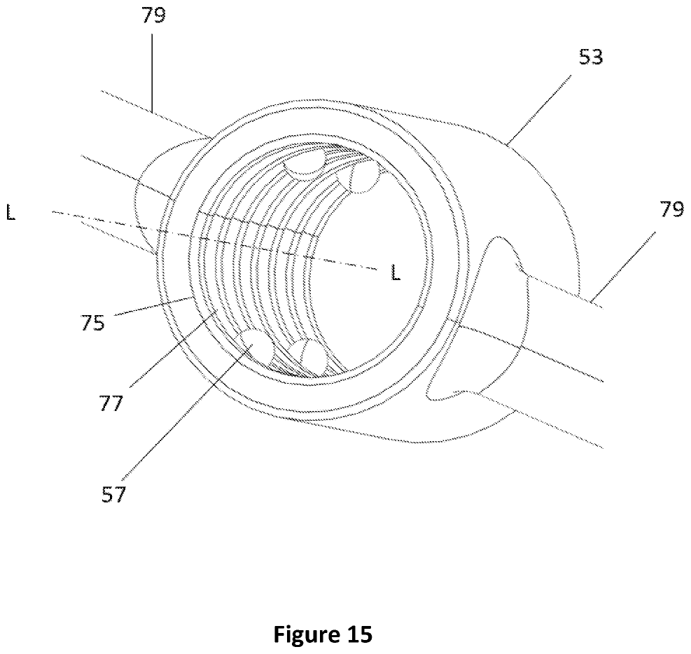

FIG. 15 is a perspective view of the drive collar;

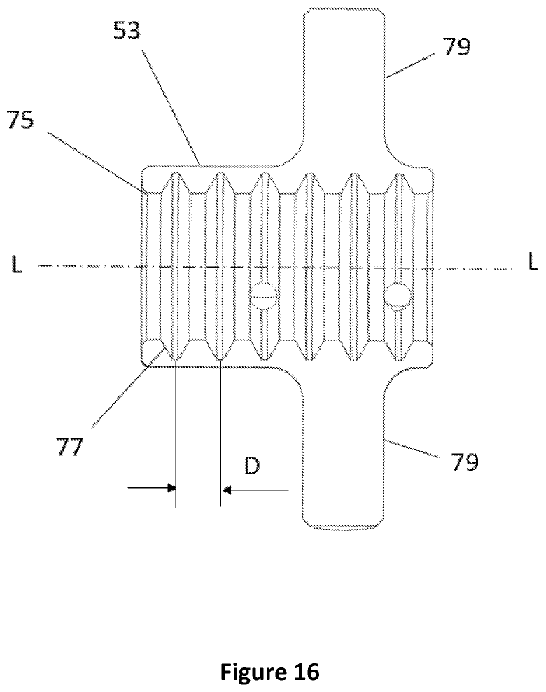

FIG. 16 is a side cross-sectional view of the drive collar showing the parallel internal grooves;

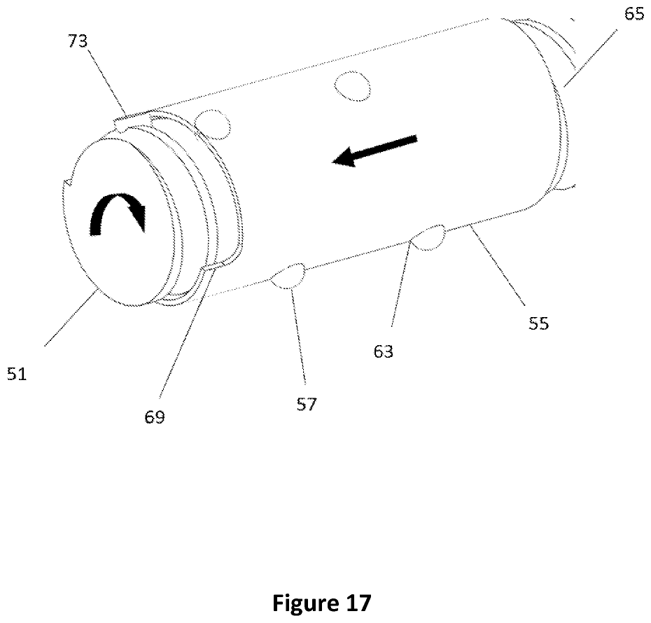

FIG. 17 is a perspective view of the ball screw and ball cage in an orientation such that the ball cage rotates freely relative to the ball screw;

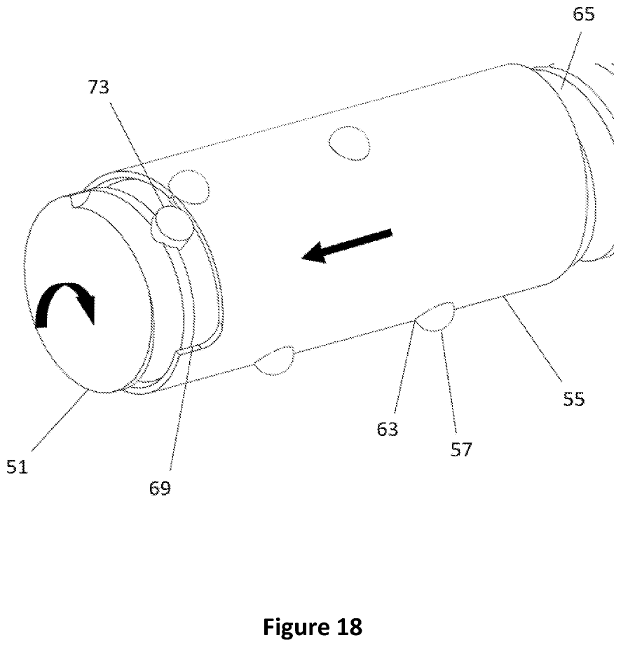

FIG. 18 is a perspective view of the ball screw and ball cage of FIG. 17 wherein the relative positions of the ball screw and ball cage have changed and an end stop is shown in close proximity to an abutment on the ball cage; and

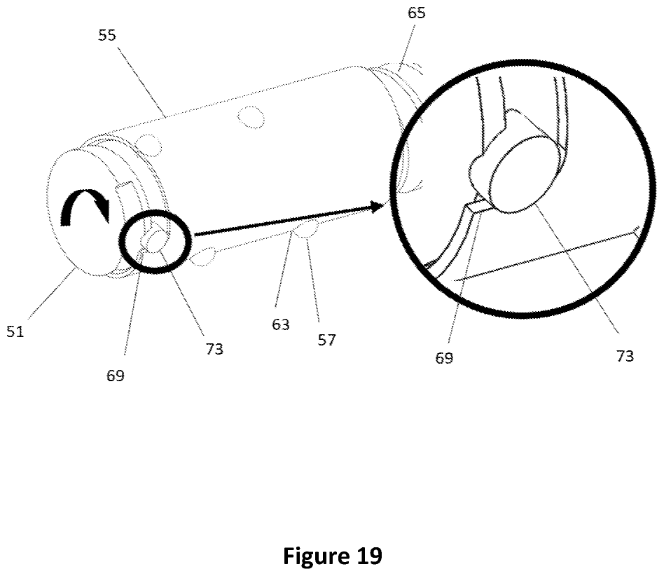

FIG. 19 is a perspective view of the ball screw and ball cage of FIG. 17 wherein the relative positions of the ball screw and ball cage have changed further and an end stop is shown in abutment with an abutment on the ball cage, as shown in the enlarged detail view.

An electromechanical digit, specifically a bionic finger 1 according to an embodiment of the present invention, is shown in FIGS. 1 and 2, as part of a prosthetic hand 3. The prosthetic hand 3 replicates a human hand and has five electromechanical digits, i.e. four bionic fingers 1 and a bionic thumb 5.

FIGS. 3 and 4 show a bionic finger 1 which is capable of articulation within a central plane CP. The bionic finger 1 comprises a base portion 7 provided with a base connector 9 which is connected to the palm 11 of the prosthetic hand 3. The base portion 7 has a socket 13 into which the proximal end of an intermediate portion 15 of the bionic finger 1 is located (the proximal end of the bionic finger is the end nearest to the palm 11). A longitudinal axis L-L is parallel to and co-planar with the central plane CP and passes through the centreline of the intermediate portion 15. A pivot pin 17 at the proximal end of the intermediate portion 15 is located within a plain bearing 19 in the base portion 7, so that an articulating joint 21, with an axis perpendicular to the central plane CP, is formed between the intermediate portion 15 and the base portion 7. A tip portion 23 of the bionic finger 1 has a socket 25 into which the distal end of the intermediate portion 15 is located. A pivot pin 27 at the distal end of the intermediate portion 15 is located within a plain bearing 29 in the tip portion 23, so that an articulating joint 31, with an axis perpendicular to the central plane CP, is formed between the intermediate portion 15 and the tip portion 23. A lateral drive arm 33 is located at each lateral side of the bionic finger 1 and substantially parallel to the central plane CP. Each lateral drive arm 33 is rotatably fixed at its proximal end to the base portion 7 and is rotatably fixed at its distal end to the tip portion 23.

FIG. 5 shows the intermediate portion 15 separated from the rest of the bionic finger 1. It has a casing 35 which is split along the central plane CP into two parts. Three casing screws 37 fix the casing 35 together.

FIG. 6 shows the tip portion 23 separated from the rest of the bionic finger 1. It has a casing 39 which is split along the central plane CP into two parts. Three casing screws 41 fix the casing 39 together.

FIG. 7 shows the bionic finger 1 with one half of the casing 35 removed from the intermediate portion 15 and one half of the casing 39 removed from the tip portion 23. Each half of the casing is provided with a depression, such that the casing 35 is hollow and provides an actuator void 43 within which are located the components of a linear actuator assembly 45. As also shown in FIGS. 8 and 9, the linear actuator assembly 45 comprises an electric motor sub-assembly 47 having a gearbox 46 (not shown) and a rotor 48 (not shown), a driveshaft 49 connected to the rotor 48, so that it rotates with it, a ball screw 51, a drive collar 53, a ball cage 55 with six identical drive balls 57, a proximal thrust washer 59 and a distal thrust washer 61. The electric motor sub-assembly 47 is constrained within the actuator void 43 so that it cannot move along the longitudinal axis L-L relative to the casing 35 and so that the electric motor sub-assembly 47 as a whole cannot rotate around axis L-L. The ball screw 51 has a driveshaft bore 62 within which the driveshaft 49 is engaged. The driveshaft 49 is keyed to the ball screw 51 by its external profile which is complementary to an internal profiled of the driveshaft bore 62. The keying of the ball screw 51 and the driveshaft 49 prevents any relative rotation between them but allows them to move axially relative to each other along axis L-L.

FIG. 10 shows the linear actuator assembly 45 with the drive collar 53, the proximal thrust washer 59 and the distal thrust washer 61 not shown, for the sake of the clarity of the drawing. The ball cage 55 is made from a thin walled cylindrical tube with a length that is greater than its diameter. The internal diameter of the ball cage 55 is slightly larger than the external diameter of the ball screw 51. The ball cage 55 is provided with six identical ball apertures 63 and within each ball aperture 63 a drive ball 57 is located. The diameter of each ball aperture 63 is slightly larger than the diameter of a drive ball 57. The ball apertures 63 are equally spaced apart along the longitudinal axis L-L of the linear actuator assembly 45 by a distance D, measured along the longitudinal axis, that is equal to the pitch P of the ball screw 51 (although the distance D can be the same as pitch P, the distance D does not have to be the same as pitch P). Starting from one end of the ball cage 55 and moving towards the other, successive ball apertures 63 are angularly displaced from each other by an angle of one hundred and twenty degrees around the longitudinal axis L-L (for example as can be seen from FIGS. 11 and 12). Each drive ball 57 sits within a ball aperture 63 and within a ball raceway 65 formed by the thread of the ball screw 51. As shown in FIG. 12 in particular, the drive balls 57 are located within the ball cage 55 such that their centre point are located at the mid-point of the thickness of the wall of the tube which forms the ball cage 55.

The ball cage 55 is provided with a proximal end stop abutment 67 at its proximal end and a distal end stop abutment 69 at its distal end. The proximal end stop abutment 67 extends outwardly from the ball cage 55 in a proximal direction that is parallel to axis L-L and by a distance that is the same as the diameter of a proximal end stop 71. The distal end stop abutment 69 extends outwardly from the ball cage 55 in a distal direction that is parallel to axis L-L and by a distance that is the same as the diameter of a distal end stop 73. The face of the proximal and distance end stop abutments 67, 69 are each perpendicular to the axis L-L and are opposed to each other circumferentially and diametrically, for example as shown in FIGS. 11 and 13.

FIG. 14 shows the linear actuator assembly 45 with the drive nut 53, the ball cage 55, the proximal thrust washer 59 and the distal thrust washer 61 not shown, for the sake of the clarity of the drawing. The drive balls 57 are shown within the ball raceway 65 of the ball screw 51 in the positions relative to each other in which they are retained by the ball cage 55. Only four of the drive balls 57 are visible because the other two are situated behind the lead screw 63. The ball raceway 65 has a semi-circular profile with a diameter that is slightly larger than the diameter of the drive balls 57, so that the drive balls 57 are a close fit within the ball raceway 65.

FIG. 15 shows the drive collar 53, without the ball cage 55 that fits within it and with only four of the drive balls 57 visible (the other two are hidden) in the relative positions that they would occupy if the ball cage 55 was in place. The drive collar 53 is made from a thick-walled cylindrical tube, with a length that is greater than its diameter. The bore 75 of the drive collar 53 is in the form of a straight sided cylinder with a diameter that is slightly greater than the diameter of the ball cage 55. A series of six V-profile grooves 77 are provided in the wall of the drive collar 53 (although the illustrated grooves have a V-profile, other profiles are envisaged, such as hemis-spherical grooves). The grooves 77 are parallel to each other, equally spaced and perpendicular to the axis L-L. The grooves are spaced from each other by a distance D (as shown in FIG. 16).

The drive collar 53 has two articulation pins 79 located on opposite external sides of the drive collar 53 that extend perpendicularly to axis L-L and that are diametrically opposed to each other. The articulation pins 79 fit within travel restraint slots 81 provided in the intermediate portion 15, for example as shown in FIG. 5. A travel restraint slot 81 is provided in each half of the casing 35 towards its distal end, the travel restraint slots 81 are parallel to the central plane CP and the longitudinal centreline of each articulation slot 81 is parallel to the axis L-L. The travel restraint slots 81 pass through the entire wall of the casing 35. The width of the travel restraint slots 81 is slightly greater than the diameter of the articulation pins 79.

The articulation pins 79 also locate within articulation slots 83 of the tip portion 23 of the bionic finger 1, as shown in FIG. 6. The articulation slots 83 have a width that is greater than the diameter of the articulation pins 79 and the articulation slots 83 pass through the wall of the casing 39 of the tip portion 23. The articulation slots are parallel to the central plane CP and are angled relative to a longitudinal axis X-X of the tip portion 23. When the bionic finger 1 has been assembled the ends of the articulation pins 79 are flush with the external surface of the casing 39 of the tip portion 23.

The dimensions of the ball screw 51, the drive collar 53, the ball cage 55, the travel restraint slots 81 and the articulation slots 83 are selected to provide the desired extent of articulation of the tip portion 23 relative to the intermediate portion 15, for example from the fully extended, or fully open, position illustrated in FIG. 3 to the fully retracted, or fully closed, position illustrated in FIG. 4. In an embodiment of the present invention the ball screw 51 has an external diameter of 4 mm, the pitch of the thread of the helical drive ball raceway 65 is 1 mm and the external diameter of the drive collar 53 is 8 mm.

In use, the tip portion 23 of the bionic finger 1 is articulated relative to the intermediate portion 15 by providing an electric current to the electric motor sub-assembly 47 and driving the electric motor (not shown) in either a clockwise or an anti-clockwise direction (when viewed from its proximal end, i.e. looking from the electric motor sub-assembly 47 towards the drive collar 53). Driving the electric motor in a clockwise direction causes the bionic finger 1 to move towards its fully retracted position and driving the electric motor in an anti-clockwise direction causes the bionic finger 1 to move towards its fully extended position.

Rotation of the electric motor causes the driveshaft 49 to rotate in the same rotational direction and thus causes the ball screw 51 to also rotate in the same direction, because the ball screw 51 is keyed to the driveshaft 49. The ball screw 51 is engaged with the drive collar 53 by means of the drive balls 57 that are contained within the ball cage 55. The helical arrangement of the ball raceway 65 of the ball screw 51 carries the drive balls 57 in an axial direction as the ball screw 51 rotates. Each drive ball 57 is simultaneously engaged with a V-profile groove 77 in the drive collar 53 and with the ball raceway 65 of the ball screw 51. The drive collar 53 cannot rotate because the articulation pins 79 are located within the travel restraint slots 81 and therefore the drive collar 53 is moved axially along axis L-L.

The degree of travel of the drive collar 53 is determined by the distance between the proximal end stop 71 and distal end stop 73 that are provided on the ball screw 51, as will be explained in further detail below. Therefore, rotation of the ball screw 51 causes the drive collar 53 to move along axis L-L. If the ball screw 51 rotates anti-clockwise then the drive collar 53 moves away from the motor sub-assembly 47, if the ball screw 51 rotates clockwise then the drive collar 53 moves towards the motor sub-assembly 47 because the drive collar 53 is located between the proximal and distal end stops 71, 73.

The ball cage 55 moves axially with the drive collar 53, because it is constrained to do so as a result of the drive balls 57 being located in the parallel V-profile grooves 77 in the drive collar 53. The ball cage 55 rotates relative to the ball screw 51 and relative to the drive collar 53.

FIGS. 17, 18 and 19 illustrate the operation of the linear actuator assembly 45 as it nears and then reaches the distal end stop 73. The drive collar 53 has been omitted from the figures for the sake of clarity. The ball screw 51 is being driven by the motor sub-assembly 47 so that it rotates anti-clockwise and so that the ball cage 55 moves axially towards the end of the ball screw 51 (whilst also rotating relative to the ball screw 51). FIG. 17 shows the distal end stop abutment 69 of the ball cage 55 approaching the distal end stop 73 of the ball screw 51, i.e. the ball cage 55 (and thus the drive collar 53) is nearing the end of its travel in a distal direction and is still moving axially. FIG. 18 shows the relative positions of the ball cage 55 and the ball screw 51 after a further quarter rotation of the ball screw 51, such that the distal end stop 73 is nearly in contact with the distal end stop abutment 69. FIG. 19 shows the distal end stop 73 in contact with the distal end stop abutment 69. When the distal end stop 73 contacts the distal end stop abutment 69 it is no longer possible for the ball cage 55 to rotate relative to the ball screw 51. Consequently, the ball cage 55 and the ball screw 51 rotate together with the result that there is no force acting on the drive collar 53 to move it in an axial direction. This is because the drive balls 57 remain in the same place in the ball raceway 65 and so there is no longer any axially directed force imparted to the drive balls 57 by the helical ball raceway 65. The ball screw 51, the drive ball cage 55 and the drive balls 57 rotate about the longitudinal axis L-L inside the drive collar 53. The drive balls 57 run within the V-profile grooves 77 of the drive collar 57. The drive collar 53 does not move in an axial direction in the absence of any axial force imparted by the drive balls and thus comes to a halt.

The operation disclosed above with reference to FIGS. 17, 18 and 19 works in reverse when the ball screw 51 is driven clockwise. The drive collar 53 can be driven from the distal end stop 73 to the proximal end stop 71.

1. A bionic digit comprising an intermediate portion, a tip portion and a hinge connecting the tip portion to the intermediate portion, and further comprising a linear actuator assembly located within the intermediate portion that is connected to the intermediate portion and to the tip portion and that is provided with a force generator, to which is connected a rotary drive shaft, and a ball screw connected to the rotary drive shaft for rotation therewith, wherein the ball screw has a helical drive ball raceway extending around its external surface, along at least part of its length, a plurality of drive balls, each drive ball located within the helical drive ball raceway and within a drive ball aperture of a ball retention element that is located around the ball screw and that is moveable relative to the ball screw, wherein each drive ball is also located within an annular groove of a drive collar that is positioned around the ball retention element, the drive collar being rotatable relative to the ball retention element and the ball screw around the longitudinal axis L-L of the ball screw, wherein the drive collar has multiple annular grooves that are parallel to each other and perpendicular to the longitudinal axis of the ball screw, wherein the drive collar is provided with a first engagement element and the tip portion is provided with a second engagement element and the first engagement portion and the second engagement portion are engaged with each other.

2. The bionic digit as claimed in claim 1, wherein the ball screw is provided with a proximal end stop and a distal end stop and wherein the ball retention element is provided with a proximal end stop abutment and a distal end stop abutment.

3. The bionic digit as claimed in claim 1, wherein the ball screw has a circular cross-sectional profile, the ball retention element is a cylindrical tube with an internal diameter that is larger than the external diameter of the ball screw and the drive collar has a circular cross-section bore with an internal diameter that is larger than the external diameter of the ball retention element.

4. The bionic digit as claimed in claim 1, wherein the helical drive ball raceway has a hemi-spherical cross-sectional profile, wherein when a drive ball is placed within the drive ball raceway there is a clearance between at least some portion of the drive ball and the drive ball raceway.

5. The bionic digit as claimed in claim 1, wherein the multiple annular grooves of the drive collar have a V shaped cross-sectional profile and wherein the angle of the V is such that when a drive ball is placed in an annular groove at least a portion of the drive ball extends past the open end of the annular groove.

6. The bionic digit as claimed in claim 1, further comprising a base portion attached to the proximal end of the intermediate portion by a base hinge.

7. The bionic digit as claimed in claim 1, wherein the pitch of the ball screw is 0.25 mm to 4 mm.

8. The bionic digit as claimed in claim 1, wherein the force generator of the linear actuator assembly is an electric motor and gearbox with a rotor that rotates around an axis coaxial with the longitudinal axis L-L, wherein the driveshaft is attached to the rotor and rotates with the rotor and extends outwardly from the electric motor along longitudinal axis L-L, the driveshaft is located within a recess in the ball screw so that the ball screw can move axially relative to the driveshaft along axis L-L but cannot rotate relative to the driveshaft, the ball screw has a circular cross-section, the retention element is a straight-sided cylindrical tube with a plurality of circular drive ball apertures that pass through the wall of the tube and that are equally angularly spaced apart around the circumference of the tube, wherein the drive collar has a circular internal bore provided with a plurality of annular grooves that are located perpendicularly to longitudinal axis L-L, wherein the first engagement element of the drive collar is a peg that extends perpendicularly from the external surface of the drive collar and perpendicularly to a central plane CP through which longitudinal axis L-L passes, the first engagement element being located within the second engagement element which takes the form of a slot in the proximal end of the tip portion.

9. The prosthetic hand comprising a plurality of bionic digits according to claim 1, wherein, the prosthetic hand is provided with a palm, the bionic digits are each provided with a base portion and the base portion of each bionic digit is attached to the palm.
